(19) United States
(12) Patent Application Publication
(10) Pub. No.: US 2006/0013807 A1
(43) Pub. Date: Jan. 19, 2006

(54) Rapidly disintegrating enzyme-containing solid oral dosage compositions

(76) Inventors: William J. Chapello, Atlantic Highlands, NJ (US); Junfu Huang, Brampton (CA)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/889,861

(22) Filed: Jul. 13, 2004

(51) Int. Cl. A61K 38/47; A61K 9/20

(52) U.S. Cl. 424/94.61; 424/464

(57) ABSTRACT

The present invention relates to rapidly disintegrating solid oral dosage forms having an effective amount of an enzyme and a superdisintegrant.

### FIELD OF THE INVENTION

The present invention relates to rapidly disintegrating enzyme-containing solid oral dosage compositions having improved performance via improved disintegration time. More particularly, the present invention relates to solid compressed oral dosage forms having an effective amount of an enzyme and at least one superdisintegrant.

### BACKGROUND OF THE INVENTION

Enzymes have long been known to aid in the digestion of food, e.g., plant and meat, in mammals. Some mammals, particularly humans, lack effective amounts of enzymes to digest various foodstuffs.

For example, some humans are considered lactose intolerant because their bodies do not produce an effective amount of β-galactosidase, i.e, lactase, to convert lactose, a dissacharide carbohydrate found in milk produced by various animals, into glucose and galactose. Such humans may find relief from the symptoms of lactose intolerance by ingesting a solid oral dosage form containing an effective amount of β-galactosidase. As a result, there has been demand for a dietary supplement containing lactose hydrolyzing lactase enzymes

Additionally, some humans are known to have difficulty digesting sugars found in foods including legumes and cruciferous vegetables. Such humans have gas and bloating as a result and some have found relief by ingesting a solid oral dosage form containing an effective amount of α-galactosidase. As a result, there has been demand for a dietary supplement containing α-galactosidase.

Oral dosage forms useful for treating or controlling lactose intolerance have long been known. See, for example, U.S. Pat. No. 3,627,583.

Lactose hydrolyzing lactase enzymes, i.e., lactase and β-galactosidase, are known to be produced by various yeasts, bacteria and fungi. Among the organisms heretofore disclosed as useful for this purpose are yeasts, such as, Saccharomyces fragilis, Torula cremoris and Torula utilis, bacteria, such as, Escherichia coli and Lactobacillus bulgaricus, fungi, such as, Aspergillus oryzae, Aspergillus flavus and Aspergillus niger, and various other microorganisms, such as, those described in U.S. Pat. Nos. 2,681,858, 2,781,266, and 2,809,113. The lactase enzyme preparations produced by these organisms generally have pH optimums on the alkaline side or in the weakly acid pH range of about 5-7. Yeasts, which are the primary source of commercial lactases, are known to produce lactases having pH optimums of about 7.

It is also known that an acidic environment, like that found in the stomach, inactivates or destroys typical lactase enzymes. Such destruction in activity reduces the efficacy and potency of oral dosage forms formulated using typical lactase enzyme preparations. Attempts to address this problem have focused on the lactase enzyme used, e.g., the environmental pH used to grow the lactase producing fungi or yeast.

Methods of harvesting lactase from fungi or yeast grown in acidic environments are described in an attempt to overcome the inactivation and destruction of lactase activity in acid environments. See, for example, U.S. Pat. Nos. 3,629,073; 3,718,739; and 3,919,049.

Others have attempted to overcome problem of lactase inactivation by pH by using at least two different lactase enzymes, each being active at a different pH. See, for example, U.S. Pat. Nos. 6,410,018; 6,428,786; 6,562,338; and 6,562,339.

While the above solutions may provide lactase having increased efficacy and potency in an acidic environment, there is still a lag time for the lactase to actually be available in the stomach.

It has been surprisingly found that typical enzyme preparations, including those having enzymes active at high pH, have increased potency and efficacy when the amount of time such lactase enzymes are available in the stomach is increased.

However, evaluation of commercial solid dosage forms containing lactase in a simulated stomach model revealed conversion of lactose was not maximized for the products. For example, all solid dosage forms having 9000 FCC units of lactase per dosage form had long disintegration times and low total conversion. Conversely, use of lactase in solution exhibited conversions that exceeded all products. What is needed, therefore, is a new formulation for a solid lactase composition that is as small as possible, for ease of swallowing and disintegrates as soon as possible.

It has been surprisingly found that it is possible to decrease the size of solid lactase compositions while at the same time reducing disintegration times and hydrolyzing more lactose.

### SUMMARY OF THE INVENTION

The present invention relates to a rapidly disintegrating solid oral dosage form for controlling lactose intolerance in a patient in need thereof, the dosage form comprising, consisting essentially of, and/or consisting of, an effective amount of lactase and an effective amount of a plurality of superdisintegrants.

The present invention also relates to a rapidly disintegrating oral dosage form for controlling lactose intolerance in a patient in need thereof, the dosage form comprising, consisting essentially of, and/or consisting of an effective amount of lactase, an effective amount of a plurality of superdisintegrants, and an effective amount of a lubricant to aid compression, wherein the oral dosage form hydrolyzes at least 6 grams of lactose at a pH of at most about 6 in at least about 6 minutes.

The present invention further relates to a rapidly disintegrating oral dosage form for controlling lactose intolerance in a patient in need thereof, the dosage form comprising, consisting essentially of, and/or consisting of, about 9000 FCC units lactase in an amount of about 17 wt % of the oral dosage form, a combination of croscarmellose and crospovidone in an amount of about 10 wt % of the oral dosage form, wherein the oral dosage form hydrolyzes at least 6 grams of lactose at a pH of at most about 6 in at least about 6 minutes.

### DETAILED DESCRIPTION

As used herein, all numerical ranges provided are intended to expressly include at least all numbers that fall between the endpoints of ranges.

As used herein, the term “rapidly disintegrating” means disintegration according to USP 701 in six minutes or less.

Lactase

Suitable lactase for use herein include, a lactase isolated from Saccharomyces lactis, by Gist-Brocade in Delft, Holland, and sold by Enzyme Development Corporation, New York, N.Y.; a lactase from Aspergillus oryzae, Lactase Y-400, produced by K. K. Yakult Honsha; a lactase from Aspergillus oryzae, Plexazym LA 1, produced by Roehm GmbH; a lactase from Aspergillus oryzae, produced by Shinnihon Kagaku Kogyo Co.; a lactase from Kluyveromyces fragilis, produced by Sturges Enzymes, Selby, North Yorkshire, England; a lactase from Aspergillus oryzae, Takamine lactase, produced by Miles Laboratories, Inc., Elkhart, Ind.; Amano Enzyme USA Co. Ltd., Elgin, Ill. as AMANOF100 concentrate, and a lactase from Kluyveromyces fragilis produced by Novo Enzymes, Bagsvaerd, Denmark. These suppliers and others offer, generally, lactase enzyme products, including a diluent, having a potency of between 14,000 and 150,000 FCC lactase units/gram.

The above lactase enzymes are commercially produced as biological products that have variability in potency from batch to batch. Such variability requires adjustment with a diluent in order to produce a uniform commercial product. When lactase is referred to herein, it is such an admixture that is referred to.

Diluents are typically substances added to the purified lactase that provide bulk in order to produce a lactase enzyme product of the target potency. Typically, diluents for lactase enzyme products are selected from any inert pharmaceutical excipient, including, sugars, starches, cellulose and inorganic salts. More particularly, typical excipients include dextrose, mannitol, calcium phosphate, sodium citrate and microcrystalline cellulose.

Ideally, the lactase tablets herein should contain sufficient lactase to satisfy the dosage requirement of most individuals requiring the dietary supplement of lactase in most situations. Alternately, the lactase tablets herein should contain a fraction of such a dose so that the gamut of affected individuals can closely match their dosage requirements with the administration of one, two or three tablets.

The concentration of enzyme in the tablet, pill, or caplet is the governing factor of disintegration time that must be controlled. This allows minimizing the pill size for the dosage.

In a solid dosage form of the present invention, the amount of lactase used is from about 5 to about 30 wt % of the compressed dosage form, or from about 7 to about 25 wt % of the final dosage form, or from about 10 to about 15 wt % of the final dosage form or about 10 wt % of the final dosage form.

In a solid dosage form of the present invention, the amount of lactase used is from about 2550 to about 10350 FCC lactase unit/dosage form. A solid dosage form herein can contain about 9000 FCC lactase unit/dosage form and, in another embodiment, about 4500 FCC lactase unit/dosage form and in yet another embodiment, about 3000 FCC lactase unit/dosage form. It has been found useful to include lactase in an amount of from about 15 to about 40 wt % of the oral dosage form.

Other Enzymes:

While it is believed that any enzyme having efficacy in the stomach may be used in this invention, this invention is particularly useful for acid-labile enzymes, e.g., certain lactase enzymes. Other enzymes may benefit from this invention as well. For example, digestive enzymes such as proteases, amylases, lipases, cellulases, and the like may be used in formulations according to the present invention. In particular, alpha-galactosidase has been found to improve digestion of sugars found in foods including legumes and cruciferous vegetables and reduce effects generally associated with the foods, such as gas and bloating. Thus, alpha-galactosidase is a particular enzyme useful in the present invention.

Disintegrants

Disintegrants are components that aid in breaking up or disintegration of a compressed solid dosage form after oral administration. Conventional disintegrant components can be used in the present invention. Such disintegrants include starches, clays, celluloses, algins, gums, sugar alcohols, e.g., mannitol, cross-linked polymers, and low-substituted hydroxypropylcellulose (L-HPC). Starches useful include common starch (e.g., corn starch) or a pregelatinized starch.

It is also known that some materials, such as, microcrystalline cellulose (e.g., Avicel PH101®. and Avicel PH102®, described in U.S. Pat. Nos. 2,978,446; 3,141,875 and 3,023,104) present binding and disintegrating characteristics and therefore are useful both as a binder and a disintegrant. Swelling is one of the decisive factors influencing the disintegrant behavior of compacted solid pharmaceutical dosage forms and consequently the biopharmaceutical profile of drug substances.

In a solid dosage form of the present invention, the amount of disintegrant used is from about 50 to about 85 wt % of the solid dosage form, or from about 60 to about 80 wt % of the final dosage form, or from about 75 to about 80 wt % of the final dosage form or about 77 wt % of the final dosage form. The foregoing amounts include the amount of superdisintegrant in the solid dosage form.

Superdisintegrants

Superdisintegrants are pharmaceutical excipients within a larger class of excipients known as disintegrants, discussed above. Disintegrants are typically hydrophilic polymers of either natural or synthetic origin. Superdisintegrants are disintegrants that swell upon contact with water. Superdisintegrants are known to swell to at least double their non-hydrated volume on contact with water and also provide channels for water to travel upon contact. While no particular superdisintegrant is critical to the present invention, exemplary superdisintegrants include, but are not limited to:
cross-linked polyvinyl pyrollidone (crospovidone);
cross-linked carboxymethyl cellulose (croscarmellose sodium), e.g., Ac-Di-Sol.®. (which is the acronym of Accelerated Dissolution) (FMC Corporation) is an internally cross-linked form of sodium carboxymethylcellulose; and
cross-linked starch (sodium starch glycolate), e.g, Explotab®, a cross-linked, low-substituted carboxymethyl ether of poly-α-glucopyranose obtained by the suitable treatment of potato starch and has a median particle size in the range of 35-55 μm. About 25% of the glucose units are carboxymethylated.

A typical 9000 FCC lactase unit/caplet product typically have an aqueous disintegrate of 16 minutes or longer in accordance with USP 701. It is, however, an object of the present invention to decrease the disintegration time of 9000 FCCU lactase activity single dose pill to less than 6 minutes, preferably less than 2 minutes. It is believed that such disintegration time confers improved conversion of lactose of certain forms of lactase compositions, e.g., tablet, capsule, and the like.

In a solid dosage form of the present invention, the amount of superdisintegrant used is from about 5 to about 15 wt % of the solid dosage form or about 10 wt % of the final dosage form.

Binders

Binders, or granulators, are components that add cohesive properties to powdered materials, which help to ensure the dosage form remains intact after compress, as well as improving the free-flowing qualities. Binders include starch, gelatin, and sugars, such as sucrose, glucose, and dextrose, acacia, sodium alginate and the like.

Lubricants

When a solid dosage form, such as, a tablet, is made by compaction, a composition is subjected to pressure from a punch and dye. Some excipients and active ingredients have a tendency to adhere to the surfaces of the punch and dye, which can cause the product to have pitting and other surface irregularities. Lubricants can be added to the composition to aid in providing enhanced release properties.

The lubricant component can be any pharmaceutically acceptable lubricant. Such lubricants include silicone fluids, hydrogenated vegetable oils, microfine silica, stearate salts, stearic acid, polyethylene glycol, talc, sodium benzoate, sodium acetate, magnesium carbonate, magnesium oxide, and the like. The lubricant is present in an amount sufficient to provide acceptable die release properties to the tablets. Preferrably, the lubricant is present in the lactase solid dosage formulation in an amount of from about 0.25% to about 6% by weight of the solid dosage form, from about 0.5% to about 4% by weight of the solid dosage form, and about 0.5% to about 1.0% by weight of the solid dosage form.

Most lubricants are hydrophobic. The mix, blending, time with the lubricant should be sufficient to mix the ingredients and to prevent binding in the press resulting in high ejection pressures, but not excessive as the coating of the excipients with hydrophobic lubricants increases disintegration time of the solid oral dosage form.

Glidants

Glidants can be added to improve the flow properties of a solid composition and improve the accuracy of dosing. Excipients that may function as glidants include, but are not limited to, colloidal silicon dioxide, magnesium trisilicate, powdered cellulose, starch, talc and tribasic calcium phosphate.

Miscellaneous

Flavoring agents and flavor enhancers make the dosage form more palatable to the patient. Common flavoring agents and flavor enhancers for pharmaceutical products that may be included in the dosage forms of the present invention include, but are not limited to, maltol, vanillin, ethyl vanillin, menthol, citric acid, fumaric acid ethyl maltol, and tartaric acid.

The dosage forms may also be colored using any pharmaceutically acceptable colorant to improve their appearance and/or facilitate patient identification of the product and unit dosage level. The colors used may be lake or dye.

In preparing the lactase tablets of this invention, the active lactase is simply mixed with the other ingredients and pressed into tablets by standard techniques. To achieve the benefit of the invention herein, it is not critical that any order of mixing be observed. Blending can be done in a v-blender and a bin blender. It can be done in any commercially available equipment provided the uniformity of the resulting blend is at least 95% uniform. Tablet pressing can be done by any tableting machine used in the art, including a Manasty Betapress.

All dosages of the present invention can be minimized in weight and still deliver rapid disintegration times. While not critical to the present invention, the range of weights for the solid oral dosage form of the present invention can be from about 150 mg to about 1000 mg, including 190 mg to about 430 mg. Typically, the 3000 FCC lactase unit/tablet products are about 350 mg and the 4500 FCC lactase unit/tablet caplet products are about 440 mg.

The solid lactase composition of the present invention can be any solid form, including, but not limited to, tablet, caplet, dragee, capsule, pill, and pellet.

Disintegration of the solid dosage forms for the present invention can be determined using the methods and procedures described in USP 701 and USP 701 with inserts. Typically, disintegration times of the present invention are about 6 minutes or less. More preferably, the disintegration times are about 2 minutes or less.

The rapidly disintegrating solid oral lactase dosage form is administered to the patient in need thereof prior to, or concurrently with, the consumption of lactose-containing food products.

Although the invention is illustrated and described above with reference to specific embodiments, the invention is not intended to be limited to the details shown. Rather, various modifications may be made in the details within the scope and range of equivalents of the claims and without departing from the invention. It is expressly intended, for example, that all ranges broadly recited in this document include within their scope all narrower ranges and specific numbers that fall within the broader ranges.

### EXAMPLES

The following examples are intended to illustrate the invention herein and are in no way intended to be limiting:

### Example 1

The following raw materials were used to produce mixtures suitable for compressing into tablets:

| Material | Product Name | Supplier |
|---|---|---|
| Lactase enzyme | F100 concentrate | AMANO ENZYME USA |
| Croscarmellose, Sodium | AcDiSol | FMC |
| Crospovidone | Polyplasdone XL | International Specialty |
| | | Products |
| Microcrystalline | Avicel PH 200 | FMC |
| cellulose | | |
| Microcrystalline | Avicel PH 102 | FMC |
| cellulose | | |
| Mannitol | Mannogem 2080 | SPI Pharma |
| Colloidal Silicon | Cab-O-Sil M5P | Cabot Corporation |
| dioxide | | |
| Magnesium Stearate | HYQUAL #6504 | Mallinckrodt |

Ten (10) compositions containing some or all of the above materials were made. The blends of the materials were made using a 8 L v-blender or a 10 L bin blender. Loading was performed by loading in descending order of weight, half of each excipient except the lactase enzyme and magnesium stearate. The lactase was added in the middle and the rest of the excipient added in ascending order of weight, except for the magnesium stearate. The blender was then mixed for 20 minutes. The magnesium stearate was then added and blended further 2 to 5 minutes longer.

Tablets were pressed using Manasty Betapress with either 4 dies at 40 rpm or 2 dies at 70 rpm. Minimum or no precompression was used. Dies sizes included were:

| Width | Length |
|---|---|
| (inches) | (inches) |
| 0.197 | 0.416 |
| 0.235 | 0.538 |
| 0.255 | 0.570 |

Disintegration tests were done following USP 701 and USP 701 with inserts.

### Example 2

| Component | Control | Test 1 | Test 2 | Test 3 |
|---|---|---|---|---|
| Lactase | 37.91% | 37.91% | 37.91% | 37.91% |
| Croscarmellose | — | 5.00% | 15.00% | 5.00% |
| Crospovidone | — | — | — | 5.00% |
| Avicel | 61.29% | 56.29% | 20.00% | 20.00% |
| Mannitol | — | — | 26.49% | 31.49% |
| Colloidal Silicon Dioxide | 0.30% | 0.30% | 0.30% | 0.30% |
| Magnesium Stearate | 0.50% | 0.50% | 0.30% | 0.30% |
| Total | 100.00% | 100.00% | 100.00% | 100.00% |
| Dose weight | 190 mg | 190 mg | 190 mg | 190 mg |
| Last Disintegration | 32.0 | 28.6 | 16.3 | 16.0 |
| Time (minutes) | | | | |

All % given are wt %.

Typically, superdisintegrants are recommended to be used at levels of about 2 wt % or less. However, the above data indicates that it is possible to reduce disintegration time about 10% by more than doubling the amount of recommended superdisintegrant used in the solid oral dosage form. Reductions of up to about 50% of the disintegration time were found using from about 10 to about 15 wt % of the solid dosage form of superdisintegrant.

### Example 3

| Component | Test 4 | Test 5 | Test 6 | Test 7 | Test 8 | Test 9 |
|---|---|---|---|---|---|---|
| Lactase | 37.91% | 16.75% | 18.47% | 20.58% | 23.24% | 24.84% |
| Croscarmellose | 5.00% | 5.00% | 5.00% | 5.00% | 5.00% | 5.00% |
| Crospovidone | 5.00% | 5.00% | 5.00% | 5.00% | 5.00% | 5.00% |
| Avicel | 20.00% | 26.52% | 25.87% | 25.09% | 24.12% | 25.36% |
| Mannitol | 31.49% | 45.73% | 44.66% | 43.33% | 41.65% | 39.00% |
| Colloidal Silicon | 0.30% | — | — | — | — | 0.30% |
| Dioxide | | | | | | |
| Magnesium Stearate | 0.30% | 1.00% | 1.00% | 1.00% | 1.00% | 0.50% |
| Total | 100.00% | 100.00% | 100.00% | 100.00% | 100.00% | 100.00% |
| Dose weight | 190 mg | 430 mg | 390 mg | 350 mg | 310 mg | 290 mg |
| Last | 11.5 | 0.8 | 0.7 | 1.2 | 1.3 | 2.0 |
| Disintegration | | | | | | |
| Time (minutes) | | | | | | |

The results in Test 4 using 10 wt % led to the hypothesis that the enzyme has binding properties. The above dilutions were done to demonstrate the effects of various amounts of lactase on disintegration time.

What is claimed is:

1. A rapidly disintegrating solid oral dosage form for controlling lactose intolerance in a patient in need thereof, the dosage form comprising:
an effective amount of lactase, and
an effective amount of a plurality of superdisintegrants.

2. The oral dosage form of claim 1, wherein the effective amount of lactase is from about 2550 to about 10350 FCC units per dosage form.

3. The oral dosage form of claim 2, wherein the effective amount of lactase is about 9000 FCC units per dosage form.

4. The oral dosage form of claim 1, wherein the effective amount of superdisintegrants is from about 5 to about 15 wt % of the oral dosage form.

5. The oral dosage form of claim 4, wherein the plurality of superdintegrants are selected from a cross-linked polyvinyl pyrollidone, a cross-linked carboxymethyl cellulose, and a cross-linked starch.

6. The oral dosage form of claim 5, wherein the plurality of superdisntegrants are selected from crospovidone, croscarmellose, and sodium starch glycolate.

7. The oral dosage form of claim 4, wherein the effective amount of superdisintegrant is about 10 wt % of the oral dosage form.

8. The oral dosage form of claim 7, wherein the plurality of superdintegrants are selected from a cross-linked polyvinyl pyrollidone, a cross-linked carboxymethyl cellulose, and a cross-linked starch.

9. The oral dosage form of claim 8, wherein the plurality of superdisntegrants are selected from crospovidone, croscarmellose, and sodium starch glycolate.

10. The oral dosage form of claim 4, wherein the effective amount of lactase is from about 15 to about 40 wt % of the oral dosage form.

11. The oral dosage form of claim 10, wherein the plurality of superdintegrants are selected from a cross-linked polyvinyl pyrollidone, a cross-linked carboxymethyl cellulose, and cross-linked starch.

12. The oral dosage form of claim 11, wherein the plurality of superdisntegrants are selected from crospovidone, croscarmellose, and sodium starch glycolate.

13. The oral dosage form of claim 10, wherein the effective amount of superdisintegrant is about 10 wt % of the oral dosage form.

14. The oral dosage form of claim 13, wherein the plurality of superdintegrants are selected from a cross-linked polyvinyl pyrollidone, a cross-linked carboxymethyl cellulose, and a cross-linked starch.

15. The oral dosage form of claim 14, wherein the plurality of superdisntegrants are selected from crospovidone, croscarmellose, and sodium starch glycolate.

16. The oral dosage form of claim 15, wherein the weight of the oral dosage form is from about 150 mg to about 1000 mg.

17. The oral dosage form of claim 16, wherein the weight of the oral dosage form is about 350 mg.

18. The oral dosage form of claim 1, wherein the dosage weight is about 350 mg.

19. The oral dosage form of claim 1, wherein the oral dosage form hydrolyzes at least 6 grams of lactose at a pH of at about 6 or less in at least about 6 minutes.

20. The oral dosage form of claim 19, wherein the effective amount of lactase is from about 2550 to about 10350 FCC units per dosage form.

21 The oral dosage form of claim 20, wherein the effective amount of lactase is about 9000 FCC units per dosage form.

22. The oral dosage form of claim 21, wherein the effective amount of the plurality of superdisintegrants is from about 5 to about 15 wt % of the oral dosage form.

23. The oral dosage form of claim 22, wherein the plurality of superdintegrants are selected from a cross-linked polyvinyl pyrollidone, a cross-linked carboxymethyl cellulose, and a cross-linked starch.

24. The oral dosage form of claim 23, wherein the plurality of superdisntegrants are selected from crospovidone, croscarmellose, and sodium starch glycolate.

25. The oral dosage form of claim 22, wherein the effective amount of the plurality of superdisintegrant is about 10 wt % of the oral dosage form.

26. The oral dosage form of claim 25, wherein the plurality of superdintegrants are selected from cross-linked polyvinyl pyrollidone, croscarmellose, cross-linked carboxymethyl cellulose, cross-linked starch, and low-substituted hydroxypropylcellulose.

27. The oral dosage form of claim 26, wherein the plurality of superdisntegrants are selected from crospovidone, croscarmellose, and sodium starch glycolate.

28. A rapidly disintegrating oral dosage form for controlling lactose intolerance in a patient in need thereof, the dosage form comprising:
an effective amount of lactase,
an effective amount of a plurality of superdisintegrants, and
an effective amount of a lubricant to aid compression, wherein the oral dosage form hydrolyzes at least 6 grams of lactose at a pH of at most about 6 in at least about 6 minutes.

29. A rapidly disintegrating oral dosage form for controlling lactose intolerance in a patient in need thereof, the dosage form comprising
about 9000 FCC units lactase in an amount of about 17 wt % of the oral dosage form,
a combination of croscarmellose and crospovidone in an amount of about 10 wt % of the oral dosage form, wherein the oral dosage form hydrolyzes at least 6 grams of lactose at a pH of at most about 6 in at least about 6 minutes.
